(19) United States
(12) Patent Application Publication
(10) Pub. No.: US 2010/0022815 A1
(43) Pub. Date: Jan. 28, 2010

(54) PROCESS FOR PRODUCTION OF LOWER HYDROCARBONS AND APPARATUS FOR THE PRODUCTION

(75) Inventors: Nobuyasu Chikamatsu, Yokohama-shi (JP); Hirofumi Ito, Mito-shi (JP); Koji Ooyama, Yokohama-shi (JP); Motohisa Kume, Yokohama-shi (JP); Chizu Inaki, Ishioka-shi (JP)

Correspondence Address: DARBY & DARBY P.C., P.O. BOX 770, Church Street Station, New York, NY, 10008-0770, US

(73) Assignee: JGC CORPORATION, Tokyo (JP)

(21) Appl. No.: 12/064,508

(22) PCT Filed: Aug. 14, 2006
(86) PCT No.: PCT/JP2006/316005

§ 371 Date: Aug. 19, 2009

(30) Foreign Application Priority Data
Aug. 24, 2005 (JP) 2005-242057

(51) Int. Cl. C07C 1/00; B01J 19/00

(52) U.S. Cl. 585/640; 422/129

(57) ABSTRACT

In a process for production of lower hydrocarbons from dimethyl ether and/or methanol and an apparatus for the production, the object is; to increase the selectivity of the reaction product to thereby produce a target product at a high final yield, to prolong the catalyst lifetime, and to improve the safety in the operation of the apparatus. An apparatus to be used includes: a reactor 2 which reacts dimethyl ether and/or methanol in the presence of a catalyst to produce lower hydrocarbons; a separator 4 which separates ethylene from lower hydrocarbons from the reactor 2; and a converter 6 which converts the ethylene separated by the separator 4 into a hydrocarbon having 4 or more carbon atoms, and feeds this hydrocarbon into the upstream or downstream of the reactor 2.

### TECHNICAL FIELD

The present invention relates to a process for producing lower hydrocarbons such as propylene from dimethyl ether and/or methanol by a dehydration reaction, and an apparatus therefor.

Priority is claimed on Japanese Patent Application No. 2005-242057, filed Aug. 24, 2005, the content of which is incorporated herein by reference.

### BACKGROUND ART

Processes for producing lower hydrocarbons from dimethyl ether (hereunder, also expressed as DME) and/or methanol (hereunder, also expressed as “DME and the like”) have been conventionally developed as one of the processes for synthesizing propylene, ethylene, and the like, the demand for which is expected to expand from now on.

In these processes, dimethyl ether and/or methanol is used as a raw material, and is fed into a reactor filled with; a catalyst such as am MFI structure zeolite catalyst (refer to Japanese Unexamined Patent Application, First Publication No. H04-217928), an MFI structure zeolite catalyst containing alkaline-earth metal (refer to Japanese Unexamined Patent Application, First Publication No. 2005-138000), and a silica-aluminophosphate catalyst (refer to U.S. Pat. No. 6,534,692), followed by a reaction under conditions of; temperature from 300 to 600° C., space velocity from 0.1 to 20 g-DME/(g-catalyst·hour), and pressure from 0.1 to 100 atm, so as to obtain a reaction product of a mixture containing; lower olefins such as ethylene and propylene, paraffin, an aromatic hydrocarbon, and the like. Here, the term “space velocity” refers to the weight based hourly space velocity which is the ratio of the DME supply velocity to the weight of the catalyst.

In these production processes, a long catalyst lifetime and a high selectivity for a target hydrocarbon such as propylene in the reaction product are expected.

However, the selectivity for the target hydrocarbon is not always high, and a lot of by-products are also produced. For example, in the method disclosed in Japanese Unexamined Patent Application, First Publication No. H04-217928, the composition of hydrocarbons (weight ratio) in the reaction product with respect to the raw material comprising dimethyl ether, methanol, and water vapor becomes: paraffin (C1-C4) 5.58%, ethylene 7.27%, propylene 42.14%, butenes 25.66%, and hydrocarbons having 5 or more carbon atoms 19.35%.

For this reason, an attempt has been made to convert by-products into the target product again, so as to increase the final yield of the target product. For example, in Published Japanese translation No. 2003-535069 of PCT International Publication, it is disclosed that, among by-products, ethylene and butenes are recycled and supplied to a reactor together with dimethyl ether and/or methanol, so as to increase the final yield of propylene serving as the target product.

Moreover, in U.S. Pat. No. 6,303,839 and U.S. Pat. No. 5,914,433, although by-products are not recycled, it is shown that olefins having 4 or more carbon atoms are separately supplied to a catalytic cracking reactor to produce ethylene and propylene, so as to increase the final yield of the target product.

Furthermore, in U.S. Pat. No. 5,990,369, although by-products are not recycled, ethylene and butenes are supplied to a metathesis reactor to thereby increase the final yield of propylene.

However, these improved synthesis methods have had a drawback in that the lifetime of catalysts for converting dimethyl ether and/or methanol into lower hydrocarbons is short. Moreover, since the reaction for synthesizing lower hydrocarbons from dimethyl ether and/or methanol is an exothermic reactions there is concern that increasing the temperature of the reactor acceleratedly deactivates the catalyst, and thus care must be taken in the operation of the apparatus.

[Patent Document 1] Japanese Unexamined Patent Application, First Publication No. H04-217928

[Patent Document 2] Japanese Unexamined Patent Application, First Publication No. 2005-138000

[Patent Document 3] U.S. Pat. No. 6,534,692

[Patent Document 4] Published Japanese translation No. 2003-535069 of PCT International Publication

[Patent Document 5] U.S. Pat. No. 6,303,839

[Patent Document 6] U.S. Pat. No. 5,914,433

[Patent Document 7] U.S. Pat. No. 5,990,369

### DISCLOSURE OF INVENTION

Accordingly, an object of the present invention is; to increase the selectivity of the reaction product, to increase the final yield of the target product, to prolong the catalyst lifetime, and to improve the safety in the operation of the apparatus, when producing lower hydrocarbons from dimethyl ether and/or methanol.

A first aspect of the present invention is a process for the production of lower hydrocarbons by feeding dimethyl ether and/or methanol to a reactor to effect a reaction in the presence of a catalyst, wherein the process includes: separating ethylene from lower hydrocarbons in the reaction product; converting this ethylene into a hydrocarbon having 4 or more carbon atoms; and feeding this hydrocarbon into the upstream or downstream of the reactor.

A second aspect of the present invention is a process for the production of lower hydrocarbons by feeding dimethyl ether and/or methanol to a reactor to effect a reaction in the presence of a catalyst, wherein the process includes: separating ethylene from lower hydrocarbons in the reaction product; converting this ethylene into a hydrocarbon having 4 or more carbon atoms; and feeding this hydrocarbon into the upstream of the reactor to produce tower hydrocarbons together with the dimethyl ether and/or methanol.

A third aspect of the present invention is a process for the production of lower hydrocarbons of either one of the first and second aspects, wherein the process includes: introducing hydrocarbons having 4 to 6 carbon atoms among respective components produced from the lower hydrocarbons of the reaction product by the separation, into the upstream of the reactor without going through the conversion.

A fourth aspect of the present invention is a process for the production of lower hydrocarbons by feeding dimethyl ether and/or methanol to a reactor to effect a reaction in the presence of a catalyst, wherein the process includes: separating ethylene from lower hydrocarbons in the reaction product by a separator; converting this ethylene into a hydrocarbon having 4 or more carbon atoms by a converter; feeding this hydrocarbon into the downstream of the reactor; placing this hydrocarbon with the lower hydrocarbon of the reaction product, then feeding it into the separator; converting ethylene among respective components obtained from the separation, into a hydrocarbon having 4 or more carbon atones by the converter; and feeding hydrocarbons having 4 to 6 carbon atoms separated by the separator into the upstream of the reactor.

A fifth aspect of the present invention is a process for the production of lower hydrocarbons of any one of the first to fourth aspects, wherein the hydrocarbon produced by the conversion includes an olefin having 4 to 6 carbon atoms.

A sixth aspect of the present invention is an apparatus for the production of lower hydrocarbons, including: a reactor which reacts dimethyl ether and/or methanol in the presence of a catalyst to produce lower hydrocarbons; a separator which separates ethylene from lower hydrocarbons from the reactor; and a converter which converts the ethylene separated by the separator into a hydrocarbon having 4 or more carbon atoms, and feeds this hydrocarbon into the upstream or downstream of the reactor.

### EFFECTS OF THE INVENTION

According to the present invention, the selectivity of the reaction product is increased and the final yield of a target product such as propylene is increased. Moreover, by supplying hydrocarbons having 4 or more carbon atoms into a reactor together with dimethyl ether and/or methanol, the load on the catalyst is alleviated and the catalyst lifetime is prolonged. Furthermore, if hydrocarbons having 4 or more carbon atoms are supplied into the reactor together with dimethyl ether and/or methanol, the reaction from the hydrocarbons having 4 or more carbon atoms is comprehensively an endothermic reaction. Therefore heat due to the exothermic reaction of dimethyl ether and/or methanol is absorbed to thereby suppress the temperature increase of the reactor, to reduce the deactivation of the catalyst, and to improve the safety in the operation of the reactor. Moreover, when the conversion ratio of ethylene in the converter 6 is low, the reaction product is fed into the downstream of the reactor 2, and unreacted ethylene is again converted into a hydrocarbon having 4 or more carbon atoms by the converter. Therefore unreacted ethylene is not fed into the reactor 2, and the reduction in the catalyst lifetime can be prevented.

### BRIEF DESCRIPTION OF THE DRAWINGS

FIG. 1 is a schematic diagram showing an example of the production apparatus of the present invention.

FIG. 2 is a schematic diagram showing a flow of a conventional production process.

FIG. 3 is a schematic diagram showing a flow of a conventional production process.

FIG. 4 is a schematic diagram showing a flow of a conventional production process.

FIG. 5 is a schematic diagram showing a flow of a conventional production process.

### BRIEF DESCRIPTION OF THE REFERENCE SYMBOLS

2 Reactor
4 Separator
6 Converter

### BEST MODE FOR CARRYING OUT THE INVENTION

Hereunder is a detailed description of the present invention.

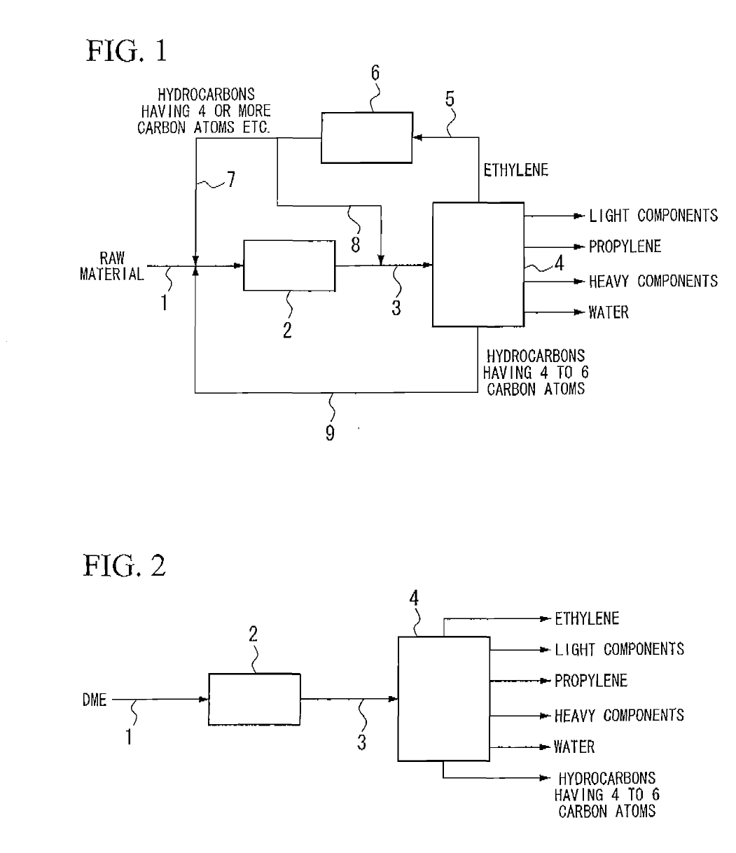

FIG. 1 shows an example of the production apparatus of the present invention.

Dimethyl ether and/or methanol serving as a raw material is fed in a gas state from a pipe 1 into a reactor 2. This raw material may also contain an additional gas such as water vapor, nitrogen, argon, and carbon dioxide.

Inside of the reactor 2 is filled with a catalyst. Due to the activity of this catalyst, a reaction such as a dehydration reaction occurs to thereby produce lower hydrocarbons having 6 or less carbon atoms such as ethylene, propylene, butene, pentene, and hexene, as the main reaction products. The abovementioned MFI structure zeolite catalyst, the MFI structure zeolite catalyst containing alkaline-earth metal, a silica-aluminophosphate catalyst, and the like are employed as the catalyst. Methods such as fluidized bed, fixed bed, old moving bed are employed.

There is no specific limitation as to the reaction condition, although it may be selected within a range of: a temperature from 300 to 600° C., a weight-based space velocity from 0.1 to 20 g-DME/(g-catalyst·hour), and a pressure from 0.1 to 100 atm.

Moreover, in this reaction, the content ratio of a target hydrocarbon in the reaction product can be changed by setting the reaction condition. For example, in order to increase the ratio of propylene, the reaction temperature is preferably set high.

The product from the reactor 2 is fed from a pipe 3 to a heat exchanger (not shown) to be cooled, and is then fed into a separator 4 to be separated into respective components such as ethylene, light components having 1 carbon atom, propylene, hydrocarbons having 4 to 6 carbon atoms, and heavy hydrocarbons having 7 or more carbon atoms.

This separator 4 uses for example, a structure comprising a plurality of distillation columns, or a structure comprising a distillation column and a separating device using a membrane or an absorption means.

Among the respective components separated by the separator 4, hydrocarbons having 4 to 6 carbon atoms are fed into the reactor 2 through a pipe 9. Ethylene separated by the separator 4 is drawn from a pipe 5 and fed into a converter 6 to be converted into hydrocarbons such as olefins having 4 or more carbon atoms. The ethylene fraction drawn from the pipe 5 may contain lower hydrocarbons such as methane or ethane and other light components without a problem.

The light components having 1 carbon atom and heavy hydrocarbons having 7 or more carbon atoms separated by the separator 4 are low in reactivity, and are thus not recycled into the reactor 2.

Inside of this converter 6 is filled with a catalyst such as a Ziegler catalyst, although the catalyst is not specifically limited, and a oligomerization reaction occurs under a reaction condition of; temperature from 45 to 55° C., weight-based space velocity from 0.1 to 10 g-ethylene/(g-catalyst·hour), and pressure from 20 to 30 atm, to convert ethylene into hydrocarbons mainly comprising olefins having 4 to 6 carbon atoms.

The hydrocarbons comprising hydrocarbons having 4 or more carbon atoms from the converter 6 are fed from the pipe 7 through the pipe 1 into the upstream of the reactor 2. Here, similarly to DME and/or methanol, the hydrocarbons having 4 or more carbon atoms that have been fed into the reactor 2 are converted into lower hydrocarbons and fed from the pipe 3 into the separator 4 to be separated into respective components in the same manner as flat of the former components.

Moreover, the structure may be such that they (hydrocarbons comprising hydrocarbons having 4 or more carbon atoms from the converter 6) are fed from the pipe 8 into the separator 4 to be separated into unreacted ethylene and hydrocarbons having 4 or more carbon atoms, and the hydrocarbons having 4 to 6 carbon atoms are fed through the pipe 9 into the upstream of the reactor 2. Furthermore, the production ratio of a specific component, such as propylene, in the produced hydrocarbons can be increased by setting the catalyst type and the reaction conditions in the converter 6, and fed from the pipe 8 to the downstream of the reactor 2, and fed into the separator 4.

In such a process for the production of lower hydrocarbons, since ethylene is separated by the separator 4 and converted into hydrocarbons having 4 or more carbon atoms by the converter 6, and then these hydrocarbons are fed into the reactor 2, the selectivity for a target product such as propylene can be increased, and the final yield of the target product can be increased.

Moreover, the catalyst lifetime for producing lower hydrocarbons from dimethyl ether and/or methanol filled in the reactor 2 is increased. The present inventors have found that the reaction when ethylene is directly recycled into the reactor 2 is mainly an exothermic reaction, thus shortening the catalyst lifetime. However, they have proven that, in contrast to where the catalyst is acceleratedly deactivated by conventional methods of directly returning ethylene from the separator 4 into the reactor 2, by sending hydrocarbons having 4 or more carbon atoms into the reactor 2 together with dimethyl ether and/or methanol such a problem does not occur, and the catalyst lifetime can be rather increased.

Therefore, the filling quantity of the catalyst can be reduced. Moreover the regeneration cycle of the catalyst can be prolonged, and the installation cost and the operation cost can be reduced.

Furthermore, since the reaction of hydrocarbons having 4 or more carbon atoms in the reactor 2 is comprehensively an endothermic reaction, the temperature increase in the reactor 2 due to the exothermic reaction of dimethyl ether and/or methanol is lessened. By feeding the hydrocarbons having 4 or more carbon atoms converted by the converter 6 into the upstream of the reactor 2, the deactivation of the catalyst is reduced and the operation of the apparatus is stabilized.

On the other hand, when the conversion ratio of ethylene in the converter 6 is low, it is not preferable to feed the reaction product into the upstream of the reactor 2, since the ethylene component enters the reactor 2 through the pipe 7, reducing the catalyst lifetime.

In this case, if the reaction product is fed into the downstream of the reactor 2, the unreacted ethylene is separated by the separator 4, and is again converted into a hydrocarbon having 4 or more carbon atoms by the converter, so that unreacted ethylene is not fed into the reactor 2.

Moreover, the hydrocarbons having 4 to 6 carbon atoms produced in the converter 6 pass through the separator 4 and are then fed into the reactor by the pipe 9, without passing through the pipe 7.

Hereunder, specific examples are shown.

### (1) Measurement of Catalyst Lifetime and Synthesis Condition

In order to reveal the effect of the difference in the process based on the recycle of byproducts on the catalyst lifetime in the synthesis of lower hydrocarbons by the dehydration reaction of dimethyl ether, the catalyst lifetime was measured as follows.

Dimethyl ether was used as a raw material, and propylene which is an olefin having 3 carbon atoms was regarded as the target product. An isothermal fixed bed reactor was used as the reactor 2, in which an MFI structure zeolite catalyst containing alkaline-earth metal (refer to Japanese Unexamined Patent Application, First Publication No. 2005-138000) was filled.

Regarding the reaction condition of the reactor 2, the temperature was made 530° C. and the pressure was made ambient pressure. The weight-based hourly space velocity (WHSV) which is the ratio of the DME supply velocity to the weight of the catalyst, was made 2.4 g-DME/(g-catalyst·hour). The flow rates of all recycled components in the system were expressed as “g-(component)/(g-catalyst·hour)” based on the weight of the catalyst filled in the reactor 2.

The weight of DME treated by 1 g of the catalyst during the time from the beginning of the reaction to the time when the conversion ratio of DME reached at most 99.9% was defined as the “catalyst lifetime”. This unit was expressed by “g-DME/g-catalyst”.

Moreover, the term “product composition” was defined as the composition (%) of components in the product based on the weight of supplied DME-containing carbon, measured by gas chromatography analysis, at the time when the reaction is stable, that is 10 to 15 hours from the beginning of the reaction.

Water produced as a by-product of the reaction was not included in the ratio of the product composition. Water produced in the following Comparative Examples and Examples was 0.94 g-H₂O/(g-catalyst·hour) in all cases.

In the reaction product, ethane, propane, and components having 1 carbon atom were regarded as light components, and benzene and hydrocarbons having 7 or more carbon atoms were regarded as heavy components. Hydrocarbons having 4 to 6 carbon atoms exclude benzene.

Based on the above preconditions, the following Comparative Examples 1 to 4 and Example 1 were performed.

### (2) Comparative Example 1

From the flow shown in FIG. 2, lower hydrocarbons were produced from dimethyl ether. In this example, by-products were not recycled.

The yields of respective components from dimethyl ether, and the catalyst lifetime, serving as the bases for the following comparative examinations, are shown. Since by-products are not recycled, the final yield of propylene is lower than that of the following respective examples as a result.

The lifetime of the catalyst filled in the reactor 2 was 610 g-DME/g-catalyst. The product composition at the outlet of the reactor 2 was: ethylene 14%, propylene 41%, hydrocarbons having 4 to 6 carbon atoms 37%, and others (light components and heavy components) 8%, on a carbon basis. The propylene yield from the raw material DME was 41% on a carbon basis. The main material balance is shown in Table 1.

TABLE 1

| | Raw material | Inlet of | Outlet of | |
|---|---|---|---|---|
| | DME | reactor 2 | reactor 2 | Final product |
| | (g/g − | (g/g − | (g/g − | (g/g − |
| | cat · h) | cat · h) | cat · h) | cat · h) |
| DME | 2.40 | 2.40 | 0.00 | 0.00 |
| Light | 0.00 | 0.00 | 0.04 | 0.04 |
| components | | | | |
| Ethylene | 0.00 | 0.00 | 0.21 | 0.21 |
| Propylene | 0.00 | 0.00 | 0.60 | 0.60 |
| C₄-C₆ | 0.00 | 0.00 | 0.53 | 0.53 |
| Heavy | 0.00 | 0.00 | 0.07 | 0.07 |
| components | | | | |
| H₂O | 0.00 | 0.00 | 0.94 | 0.94 |
| Total | 2.40 | 2.40 | 2.39 | 2.39 |

### (3) Comparative Example 2

From the flow shown in FIG. 3, lower hydrocarbons were produced from dimethyl ether. In this example, ethylene and hydrocarbons having 4 to 6 carbon atoms were recycled from the separator 4 into the reactor 2. The supply ratios of the recycled components were: 0.6 g-ethylene/(g-catalyst·hour), and 2.4 g-hydrocarbons having 4 to 6 carbon atoms/(g-catalyst·hour).

The lifetime of the catalyst filled in the reactor 2 was 459 g-DME/g-catalyst. The product composition at the outlet of the reactor 2 was: ethylene 13%, propylene 23%, hydrocarbons having 4 to 6 carbon atoms 54%, and others 9%, on a carbon basis. The propylene yield from the raw material DME was 72% on a carbon basis. The main material balance is shown in Table 2.

TABLE 2

| | Raw material | Inlet of | Outlet of | |
|---|---|---|---|---|
| | DME | reactor 2 | reactor 2 | Final product |
| | (g/g − | (g/g − | (g/g − | (g/g − |
| | cat · h) | cat · h) | cat · h) | cat · h) |
| DME | 2.40 | 2.40 | 0.00 | 0.00 |
| Light | 0.00 | 0.00 | 0.16 | 0.16 |
| components | | | | |
| Ethylene | 0.00 | 0.59 | 0.59 | 0.00 |
| Propylene | 0.00 | 0.00 | 1.05 | 1.05 |
| C₄-C₆ | 0.00 | 2.44 | 2.44 | 0.00 |
| Heavy | 0.00 | 0.00 | 0.25 | 0.25 |
| components | | | | |
| H₂O | 0.00 | 0.00 | 0.94 | 0.94 |
| Total | 2.40 | 5.43 | 5.43 | 2.40 |

### (4) Comparative Example 3

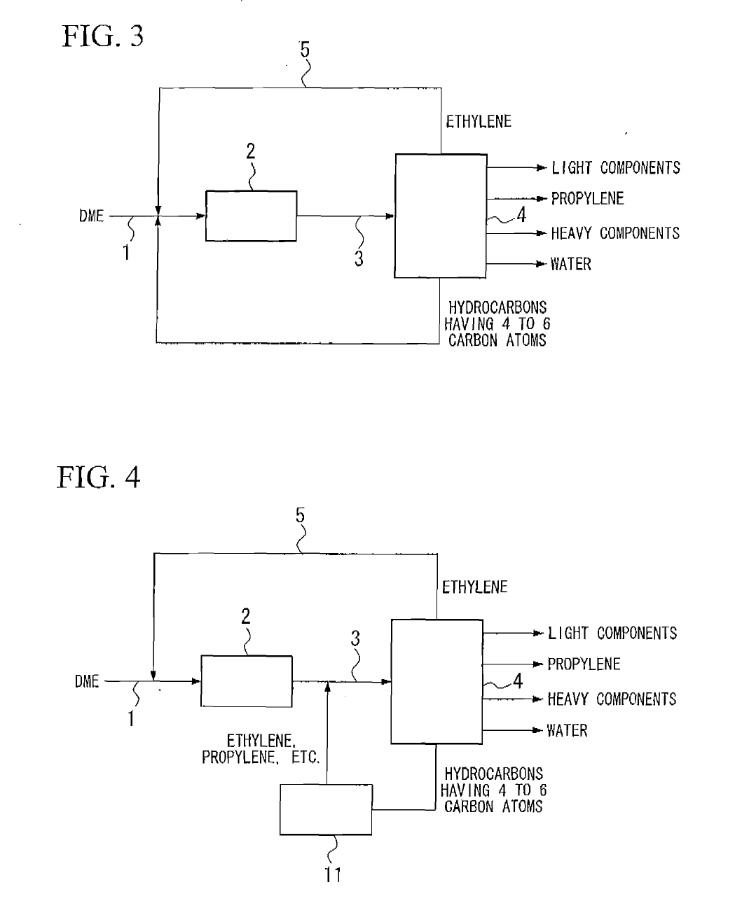

From the flow shown in FIG. 4, lower hydrocarbons were produced from dimethyl ether. This Comparative Example was based on the process proposed in U.S. Pat. No. 6,303,839, in which a catalytic cracking reactor 11 was provided, hydrocarbons having 4 to 6 carbon atoms obtained from the separator 4 were fed into the catalytic cracking reactor 11, and the product from the catalytic cracking reactor 11 was returned into the separator 4, so that recycle ethylene alone was recycled into the reactor 2. The supply ratio of the recycled component was: 0.7 g-ethylene/(g-catalyst·hour).

The lifetime of the catalyst filled in the reactor 2 was 245 g-DME/g-catalyst. The product composition at the outlet of the reactor 2 was: ethylene 25%, propylene 35%, hydrocarbons having 4 to 6 carbon atoms 34%, and others 6%, on a carbon basis. The propylene yield from the raw material DME was 72% on a carbon basis. The main material balance is shown in Table 3.

TABLE 3

| | Raw material | Inlet of | Outlet of | |
|---|---|---|---|---|
| | DME | reactor 2 | reactor 2 | Final product |
| | (g/g − | (g/g − | (g/g − | (g/g − |
| | cat · h) | cat · h) | cat · h) | cat · h) |
| DME | 2.40 | 2.40 | 0.00 | 0.00 |
| Light | 0.00 | 0.00 | 0.04 | 0.04 |
| components | | | | |
| Ethylene | 0.00 | 0.71 | 0.55 | 0.00 |
| Propylene | 0.00 | 0.00 | 0.75 | 1.05 |
| C₄-C₆ | 0.00 | 0.00 | 0.72 | 0.00 |
| Heavy | 0.00 | 0.00 | 0.10 | 0.38 |
| components | | | | |
| H₂O | 0.00 | 0.00 | 0.94 | 0.94 |
| Total | 2.40 | 3.11 | 3.10 | 2.41 |

### (5) Comparative Example 4

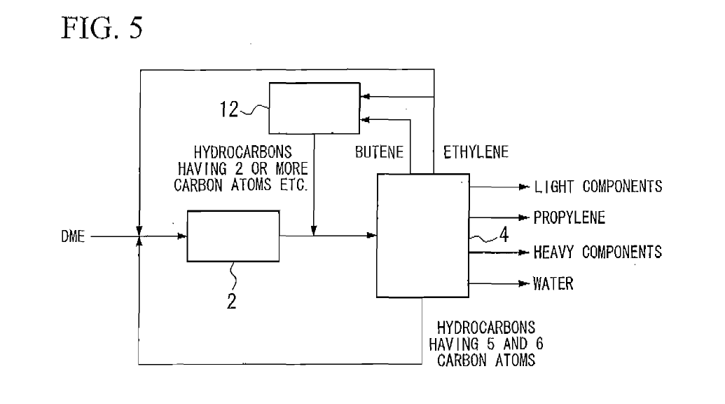

From the flow shown in FIG. 5, lower hydrocarbons were produced from dimethyl ether. This example was based on the process proposed in U.S. Pat. No. 5,990,369, in which a metathesis reactor 12 was provided, ethylene and butenes from the separator 4 were reacted in the metathesis reactor 12, and the reaction product was returned into the separator 4, so that recycle excessive ethylene with respect to butenes, and hydrocarbons having 5 or 6 carbon atoms alone were recycled into the reactor 2.

The supply ratios of the recycled components were: 0.1 g-ethylene/(g-catalyst·hour), and 0.8 g-hydrocarbons having 5 to 6 carbon atoms/(g-catalyst·hour).

The lifetime of the catalyst filled in the reactor 2 was 730 g-DME/g-catalyst. The product composition at the outlet of the reactor 2 was: ethylene 8%, propylene 23%, hydrocarbons having 4 to 6 carbon atoms 62%, and others 7%, on a carbon basis. The propylene yield from the raw material DME was 72% on a carbon basis. The main material balance is shown in Table 4.

TABLE 4

| | Raw material | Inlet of | Outlet of | |
|---|---|---|---|---|
| | DME | reactor 2 | reactor 2 | Final product |
| | (g/g − | (g/g − | (g/g − | (g/g − |
| | cat · h) | cat · h) | cat · h) | cat · h) |
| DME | 2.40 | 2.40 | 0.00 | 0.00 |
| Light | 0.00 | 0.00 | 0.09 | 0.09 |
| components | | | | |
| Ethylene | 0.00 | 0.06 | 0.25 | 0.00 |
| Propylene | 0.00 | 0.00 | 0.67 | 1.05 |
| C₄-C₆ | 0.00 | 0.78 | 1.15 | 0.00 |
| Heavy | 0.00 | 0.00 | 0.13 | 0.32 |
| components | | | | |
| H₂O | 0.00 | 0.00 | 0.94 | 0.94 |
| Total | 2.40 | 3.24 | 3.23 | 2.40 |

### (6) Example 1

From the flow shown in FIG. 1, lower hydrocarbons were produced from dimethyl ether. In this example, the converter 6 was provided, ethylene obtained from the separator 4 was mainly converted into olefins having 4 to 6 carbon atoms by the converter 6, and then the resultant product was recycled into the reactor 2 together with the raw material DME.

A reactor filled with a Ziegler catalyst was used as the converter 6. Regarding the reaction condition, the temperature was made 50° C. and the pressure was made 25 atm.

The supply ratios of the components recycled through the converter 6 were: 0.3 g-hydrocarbons having 4 to 6 carbon atoms/(g-catalyst/hour), and 0.1 g-ethylene/(g-catalyst/hour). The hydrocarbons having 4 to 6 carbon atoms obtained from the separator 4 were directly recycled, and the supply ratio was: 2.3 g-hydrocarbons having 4 to 6 carbon atoms/(g-catalyst/hour).

The lifetime of the catalyst filled in the reactor 2 was 814 g-DME/g-catalyst. The product composition at the outlet of the reactor 2 was: ethylene 9%, propylene 26%, hydrocarbons having 4 to 6 carbon atoms 55%, and others 10%, on a carbon basis. The propylene yield from the raw material DME was 72% on a carbon basis. The main material balance is shown in Table 5.

TABLE 5

| | Raw material | Inlet of | Outlet of | |
|---|---|---|---|---|
| | DME | reactor 2 | reactor 2 | Final product |
| | (g/g − | (g/g − | (g/g − | (g/g − |
| | cat · h) | cat · h) | cat · h) | cat · h) |
| DME | 2.40 | 2.40 | 0.00 | 0.00 |
| Light | 0.00 | 0.00 | 0.16 | 0.16 |
| components | | | | |
| Ethylene | 0.00 | 0.07 | 0.35 | 0.00 |
| Propylene | 0.00 | 0.00 | 1.05 | 1.05 |
| C₄-C₆ | 0.00 | 2.53 | 2.25 | 0.00 |
| Heavy | 0.00 | 0.00 | 0.25 | 0.25 |
| components | | | | |
| H₂O | 0.00 | 0.00 | 0.94 | 0.94 |
| Total | 2.40 | 5.00 | 5.00 | 2.40 |

The final propylene yields and the catalyst lifetimes in the reactor 2 in Comparative Examples 1 to 4 aid Example 1 are summarized in Table 6.

In Comparative Example 1 where by-products were not recycled, the final yield of propylene, serving as the target product was lower than that of the other examples. Thus, Comparative Example 1 is not practical. Comparing Example 1 and Comparative Example 2 where by-products were recycled, it is understood that the catalyst lifetime in Example 1 is longer in the condition where the final yields of propylene from the raw material dimethyl ether were similar to each other.

Comparing Example 1 and Comparative Examples 3 and 4 to which a process proposed in the prior patent was applied, it is understood that, in both cases, the catalyst lifetime in Example 1 is the longest in the condition where the final yields of propylene in the final product from the raw material dimethyl ether were similar to each other.

The above results show that, according to the present invention, the catalyst lifetime can be increased while maintaining a high final yield of the target product.

TABLE 6

| | | Catalyst lifetime |
|---|---|---|
| | Propylene | in reactor 2 |
| | yield *1 (%) | (g/g) *2 |
| Comparative Example 1 | 41.1 | 610 |
| Comparative Example 2 | 71.9 | 459 |
| Comparative Example 3 | 71.9 | 245 |
| Comparative Example 4 | 71.7 | 730 |
| Example 1 | 71.8 | 814 |

*1. Propylene yield in the final product from the raw material DME on a carbon weight basis.
*2. (g −DME weight treated)/(g −catalyst weight)

### INDUSTRIAL APPLICABILITY

According to the present invention, the selectivity of the reaction product is increased and the final yield of a target product such as propylene is increased. Moreover, by supplying hydrocarbons heaving 4 or more carbon atoms into a reactor together with dimethyl ether and/or methanol, the load on the catalyst is alleviated and the catalyst lifetime is prolonged. Furthermore, if hydrocarbons having 4 or more carbon atoms are supplied into the reactor together with dimethyl ether and/or methanol, since the reaction from the hydrocarbons having 4 or more carbon atoms is comprehensively an endothermic reaction, heat due to the exothermic reaction of dimethyl ether and/or methanol is absorbed to thereby suppress the temperature increase of the reactor, to reduce the deactivation of the catalyst, and to improve the safety in the operation of the reactor. Accordingly, the present invention is very useful in terms of the industry.

1. A process for the production of lower hydrocarbons by feeding dimethyl ether and/or methanol to a reactor to effect a reaction in the presence of a catalyst, comprising:
separating ethylene from lower hydrocarbons in the reaction product; converting this ethylene into a hydrocarbon having 4 or more carbon atoms; and introducing this hydrocarbon into the upstream or downstream of the reactor.

2. A process for the production of lower hydrocarbons by feeding dimethyl ether and/or methanol to a reactor to effect a reaction in the presence of a catalyst, comprising:
separating ethylene from lower hydrocarbons in the reaction product; converting this ethylene into a hydrocarbon having 4 or more carbon atoms; and introducing this hydrocarbon into the upstream of said reactor to produce lower hydrocarbons together with the dimethyl ether and/or methanol.

3. A process for the production of lower hydrocarbons of either one of claim 1 and claim 2, comprising:
introducing hydrocarbons having 4 to 6 carbon atoms among respective components produced from the lower hydrocarbons of the reaction product by said separation into the upstream of the reactor without going through said conversion.

4. A process for the production of lower hydrocarbons by feeding dimethyl ether and/or methanol to a reactor to effect a reaction in the presence of a catalyst, comprising:
separating ethylene from lower hydrocarbons in the reaction product by a separator; converting this ethylene into a hydrocarbon having 4 or more carbon atoms by a converter; introducing this hydrocarbon into the downstream of the reactor;
placing this hydrocarbon with the lower hydrocarbon of the reaction product, then introducing it into said separator; converting ethylene among respective components produced from the separation into a hydrocarbon having 4 or more carbon atoms by said converter; and
introducing hydrocarbons having 4 to 6 carbon atoms separated by said separator into the upstream of the reactor.

5. A process for the production of lower hydrocarbons of any one of claim 1 through claim 4, wherein the hydrocarbon produced by said conversion comprises an olefin having 4 to 6 carbon atoms.

6. An apparatus for the production of lower hydrocarbons, comprising: a reactor which reacts dimethyl ether and/or methanol in the presence of a catalyst to produce lower hydrocarbons; a separator which separates ethylene from lower hydrocarbons from the reactor; and a converter which converts the ethylene separated by the separator into a hydrocarbon having 4 or more carbon atoms, and feeds this hydrocarbon into the upstream or downstream of said reactor.
